(19) United States
(12) Patent Application Publication
(10) Pub. No.: US 2006/0067950 A1
(43) Pub. Date: Mar. 30, 2006

(54) Clostridial neurotoxins for use in wound healing

(75) Inventor: Harold Victor Taylor, Frankfurt (DE)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: Merz Pharma GmbH & Co. KGAA, Frankfurt am Main (DE)

(21) Appl. No.: 11/235,775

(22) Filed: Sep. 27, 2005

Related U.S. Application Data
(60) Provisional application No. 60/613,392, filed on Sep. 27, 2004.

(51) Int. Cl. A61K 39/08

(52) U.S. Cl. 424/239.1

(57) ABSTRACT

Naturally occurring and/or modified Clostridium neurotoxins, including those neurotoxins free of complexing proteins which naturally form complexes with Clostridial neurotoxins, are used to enhance healing of injured surface or superficial tissue of a patient by local administration into or in close proximity to the injured tissue. Such neurotoxins may be advantageously employed in wound healing and preventing scar formation, and find applicability in the area of ophthalmology, e.g. in treatment of injured corneal tissue, for example by closing inflamed eyes. A further embodiment includes diagnostic usage for the evaluation of effective toxin administration and medicaments for use therein.

### FIELD OF THE INVENTION

The present invention is concerned with enhancing healing of injured tissue by administering naturally occurring and/or modified Clostridium neurotoxins, and/or those neurotoxins free of complexing proteins. Such neurotoxins can be employed to enhance wound healing (including the prevention of scar formation) and find applicability in the area of ophthalmology, e.g. in treatment of injured corneal tissue, for example by closing inflamed eyes. Their diagnostic usage and medicaments for use therein are also disclosed.

### BACKGROUND OF THE INVENTION

The present invention is directed to enhancement of healing of injured surface or superficial tissue using naturally occurring and/or modified neurotoxins. Clostridium botulinum neurotoxins from serotypes A, B, C₁, D, E, F and G, and Clostridial neurotoxins free of the complexing proteins naturally occurring in Clostridial neurotoxins may be used to facilitate or enhance such healing. Clostridial neurotoxins which exhibit short duration of action, such as type E or F, may be indicated in cases where a relatively brief period of muscle paralysis is desired, such as in the treatment of wounds which heal rapidly. Clostridial neurotoxins with shorter biological persistence may exhibit reduced antibody formation, thereby maintaining the therapeutic efficacy of Clostridial neurotoxins in wound healing.

Wound healing after injury or surgical intervention may be adversely affected by tension on wound margins. Reflex muscular contractions, which may occur especially with lessening analgesia, can displace any as yet unhealed wound margins. This displacement may facilitate the entry of pathogens and, at worst, secondary healing therapy is required. In the case of secondary healing the operation wound has to be opened again and cleansed several times daily. Necrotic tissue must be removed regularly (debridement). Secondary healing takes considerably longer than primary healing: it is not only labor intensive and incurs additional cost, but also results in cosmetically unsatisfactory large scars. It can also lead to adhesions in muscles that have not been sutured together, which after healing may lead to painful random contractions of muscles scarred by connective tissue. Usually an attempt is made to counteract this by immobilizing the injured area. This may be done by, for example, applying a splint, special bandages or other devices for fixation and positioning. However, these currently used methods are in many cases inadequate and/or inconvenient and often cannot be used, especially after operation or injury to the abdomen.

The anaerobic, gram positive bacterium Clostridium botulinum produces a potent polypeptide neurotoxin, botulinum toxin, which causes a neuroparalytic disease in humans and animals referred to as botulism. The spores of Clostridium botulinum are found in soil and can grow in improperly sterilized and sealed food containers of home based canneries, which are the cause of many of the cases of botulism. The effects of botulism typically appear 18 to 36 hours after eating the foodstuffs contaminated with a Clostridium botulinum. The botulinum toxin can pass unattenuated through the lining of the gut because it is protected from the attack of pancreatic proteases by complexing proteins such as hemagglutinins and a nontoxic, nonhemagglutinating protein. The pure neurotoxin attacks peripheral motor neurons upon resorption from the gut. Symptoms of botulinum toxin intoxication can progress from difficulty walking, swallowing and speaking to paralysis of the respiratory muscles and death.

Botulinum toxin is the most lethal natural biological agent known to man. About 5-6 picograms of botulinum toxin (purified neurotoxin) serotype A (BoNT/A) given parenterally is one MLD (minimum lethal dose) in mice. One unit (U) of botulinum toxin is defined as the MLD upon intraperitoneal injection into female Swiss Webster mice weighing 18-20 grams each. Seven immunologically distinct botulinum toxin types have been characterized, these being respectively botulinum neurotoxin serotypes A, B, C₁, D, E, F and G, each of which is distinguished by neutralization with serotype-specific antibodies. The different serotypes of botulinum toxin vary in the animal species that they affect and in the severity and duration of the paralysis they evoke. For example, it has been determined that BoNT/A is 500 times more potent, as measured by the rate of paralysis produced in the rat, than is botulinum toxin serotype B (BoNT/B). Additionally, BoNT/B has been determined to be non-toxic in primates at a dose of 480 U/kg which is about 12 times the primate MLD for BoNT/A. In contrast, serotype A has a ten times longer duration of paralysis than type E when injected in mice. BoNT/C₁ acts preferentially in birds.

Botulinum toxins have been used in clinical settings for the treatment of neuromuscular disorders characterized by hyperactive skeletal muscles due to a pathological overactivity of peripheral nerves. BoNT/A has been approved by the U.S. Food and Drug Administration for the treatment of blepharospasm, strabismus and hemifacial spasm. Non-serotype A botulinum toxin serotypes apparently have a lower potency and/or a shorter duration of activity as compared to BoNT/A. Clinical effects of peripheral intramuscular BoNT/A are usually seen within one week of injection. The typical duration of symptomatic relief from a single intramuscular injection of BoNT/A averages about three months.

All the botulinum toxin serotypes apparently inhibit release of the neurotransmitter acetylcholine at the neuromuscular junction; however, they do so by affecting different neurosecretory proteins and cleaving these proteins at different sites. For example, both botulinum serotypes A and E cleave the 25 kiloDalton (kD) synaptosomal associated protein (SNAP-25); however, each toxin cleaves at a unique site within this protein. Botulinum toxin serotype C, (BoNT/C₁) has been shown to cleave both syntaxin and SNAP-25. BoNT/B, D, F and G act on vesicle-associate protein (VAMP, also called synaptobrevin), with each serotype cleaving the protein at a different site. These mechanistic differences may affect the relative potency and/or duration of action of the various botulinum toxin serotypes.

Regardless of serotype, the molecular mechanism of toxin intoxication appears to be similar and to involve several steps or stages. The intraneuronal targets of the Clostridial toxins universally participate in the process of neurotransmitter release. In the first step of the process, the toxin binds to the presynaptic membrane of the target neuron through a specific interaction between the H chain and a cell surface receptor; the receptor is thought to be different for each serotype of botulinum toxin. The carboxyl end segment of the H chain, H_{C}, appears to be important for targeting of the toxin to the cell surface.

In the second step, the toxin is engulfed by the cell through receptor-mediated endocytosis, and an endosome containing the toxin is formed. In the next step the toxin escapes the endosome into the cytoplasm of the cell. This step is thought to be mediated by the amino end segment of the H chain, H_{N}, which triggers a conformational change of the toxin in response to a pH of about 5.5 or lower. Endosomes are known to possess a proton pump which decreases intra-endosomal pH. The conformational shift exposes hydrophobic residues in the toxin, which permits the toxin to embed itself in the endosomal membrane. The toxin then translocates through the endosomal membrane into the cytosol.

The next step of the mechanism of botulinum toxin activity involves reduction of the disulfide bond joining the H and L chains. The entire toxic activity of botulinum toxins is contained in the L chain of the holotoxin which has to be separated from the heavy chain to achieve its full activity; the L chain is a zinc (Zn⁺⁺) endopeptidase which, in the last step, selectively cleaves proteins essential for recognition and docking of neurotransmitter-containing vesicles to the cytoplasmic surface of the plasma membrane, and fusion of the vesicles with the plasma membrane.

The molecular weight of the botulinum neurotoxin protein molecule, for all seven of the known botulinum toxin serotypes, is about 150 kD. However, the botulinum toxins are released by Clostridial organisms as complexes comprising the 150 kD botulinum toxin protein molecule along with associated haemagglutinins and non-toxin proteins. Thus, the BoNT/A complex can be produced by Clostridium bacteria as 900 kD, 500 kD and 300 kD forms. BoNT/B and C, are apparently produced as only a 500 kD complex. BoNT/D is produced as both 300 kD and 500 kD complexes. Finally, BoNT/E and F are produced as only approximately 300 kD complexes. The complexes (i.e. molecular weight greater than about 150 kD) are believed to contain non-toxin hemagglutinins and a non-toxin, non-toxic non-hemagglutinin protein.

Repeated injection of the complex is followed in a considerable number of patients by formation of specific neutralizing antibodies which are also directed against the neurotoxin. The direct consequence is that antibody-positive patients no longer respond to the complex. However, they might be treated with other toxin types, although not all of them are approved for therapy. When the patient has been tested with all the toxin types and has formed antibodies against them, further administration of a botulinum toxin complex (irrespective of the type) no longer provides a remedy. It must be taken into account in this connection that each dose of complex contributes to increasing the antibody titer until further administration of the complex no longer makes sense because no effect is achieved.

The formation of specific antibodies may be facilitated by the non-toxin constituents of the complex. The neurotoxin, fixed in the complex, remains in the tissue for a long period and may activate immune cells which migrate into the tissue to form antibodies. The long residence time does not result in increased uptake by the target cells, however, since poisoned target cells are no longer able to take up toxin. The neurotoxin which slowly dissociates out of the complex thus now has only immunological activity. Moreover, the non-toxin proteins present in the complex may intensify an immune response. Hemagglutinins are lectins, that is to say proteins which are distinguished by a high affinity for certain sugars. Because of their binding to sugar structures, lectins have immuno-stimulating effects. Thus, it has been possible to show that the lectins concanavalin A, phytohemagglutinin and pokeweed mitogen activate T and B lymphocytes. The hemagglutinins of the botulinum toxin complexes, which likewise bind to membrane-associated sugars, are thus able in a similar way to act as immunoadjuvants and contribute to antibody formation and thus to failure of the therapy.

An object of the present invention was therefore to develop an alternative mode of treatment for wound healing and preventing scar formation. In particular, the inventor proposes a suitable active ingredient with which patients may effectively be treated without the formation of neutralizing antibodies and with which patients who have already formed neutralizing antibodies may be treated.

In vitro studies have indicated that botulinum toxins inhibit potassium cation induced release of both acetylcholine and norepinephrine from primary cell cultures of brainstem tissue. Additionally, it has been reported that botulinum toxins inhibit the evoked release of both glycine and glutamate in primary cultures of spinal cord neurons and that in brain synaptosome preparations botulinum toxin inhibits the release of each of the neurotransmitters acetylcholine, dopamine, norepinephrine, CGRP and glutamate.

Clostridium neurotoxin may be obtained by establishing and growing cultures of Clostridium botulinum in a fermenter and then harvesting and purifying the fermented mixture in accordance with known procedures. All the botulinum toxin types are initially synthesized as inactive single chain proteins which must be cleaved or nicked by proteases to become neuroactive. The bacterial strains that produce botulinum toxin serotypes A and G possess endogenous proteases which process the toxin, and therefore, may be recovered from bacterial cultures in predominantly their active form. In contrast, botulinum toxin serotypes C₁, D, and E are synthesized by nonproteolytic strains of Clostridium and are therefore typically inactive when recovered from culture. Subsequent activation can be performed using trypsin as a peptidase. It cleaves the prominent nicking site that is exposed preferentially to the enzyme. Serotypes B and F are produced by both proteolytic and nonproteolytic strains and therefore can be recovered in either the active or inactive form. However, even the proteolytic strains that produce, for example, the BoNT/B serotype, only cleave a portion of the toxin produced. The exact proportion of nicked to unnicked molecules depends on different factors, including the length of incubation and the temperature of the culture. Therefore, any preparation of BoNT/B is likely to contain a certain percentage of inactive toxin, which may be responsible for the known significantly lower potency of BoNT/B as compared to BoNT/A.

A process for preparing neurotoxin preparations free of the associated complexing proteins is disclosed in International Patent Application No. WO 00/74703. The subject matter of this application is herein incorporated by reference. Pharmaceutical compositions comprising a botulinum neurotoxin from Clostridium botulinum, the neurotoxin being free of the complexing proteins naturally present in the botulinum neurotoxin complex, are disclosed in U.S. patent application Ser. No. 11/184,495 and corresponding PCT/US2005/025408. The subject matter of said applications, herein incorporated by reference, pertains to pharmaceutical compositions which comprise a botulinum neurotoxin from Clostridium botulinum, the neurotoxin being free of the complexing proteins naturally present in the botulinum neurotoxin complex, which pharmaceutical compositions have good stability and are advantageously formulated free of human serum albumin.

Frevert, J (DE103 33 317 and WO 2005/007185) discloses a composition for stabilizing protein active ingredients, such as Clostridial neurotoxins, in pharmaceuticals comprising: a) a surface-active substance, for example a non-ionic detergent (surfactant); and b) a mixture of at least two amino acids, selected from either Glu and Gln or Asp and Asn.

It has been reported that BoNT/A has been used in clinical settings as follows:
(1) about 75-125 units of BOTOX® per intramuscular injection (multiple muscles) to treat cervical dystonia;
(2) 5-10 units of BOTOX® per intramuscular injection to treat glabellar lines (brow furrows) (5 units injected intramuscularly into the procerus muscle and 10 units injected intramuscularly into each corrugator supercilii muscle);
(3) about 30-80 units of BOTOX® to treat constipation by intrasphincter injection of the puborectalis muscle;
(4) about 1-5 units per muscle of intramuscularly injected BOTOX® to treat blepharospasm by injecting the lateral pre-tarsal orbicularis oculi muscle of the upper lid and the lateral pre-tarsal orbicularis oculi of the lower lid;
(5) to treat strabismus, extraocular muscles have been injected intramuscularly with between about 1-5 units of BOTOX®, the amount injected varying based upon both the size of the muscle to be injected and the extent of muscle paralysis desired (i.e. amount of diopter correction desired); and
(6) to treat upper limb spasticity following stroke by intramuscular injections of BOTOX® into five different upper limb flexor muscles, as follows:
   (a) flexor digitorum profundus: 7.5 U to 30 U
   (b) flexor digitorum sublimes: 7.5 U to 30 U
   (c) flexor carpi ulnaris: 10 U to 40 U
   (d) flexor carpi radialis: 15 U to 60 U
   (e) biceps brachii: 50 U to 200 U. Each of the five indicated muscles has been injected at the same treatment session, so that the patient receives from 90 U to 360 U of upper limb flexor muscle BOTOX® by intramuscular injection at each treatment session.

One of the reasons that BoNT/A has been selected over the other serotypes, for example serotypes B, C₁, D, E, F and G, for clinical use is that botulinum toxin type A has a substantially longer lasting therapeutic effect. In other words, the inhibitory effect of botulinum toxin from serotype A is more persistent.

Alternatively, there may be a need to use short-lasting neurotoxins such as serotype E or F or modified neurotoxins which exhibit suitable effect duration.

Presently, the basis for the differences in persistence among the various botulinum toxins is unknown. However, there are two main theories explaining the differences in the persistence of the toxins. Without wishing to be bound by any theory of operation or mechanism of action, these theories will be discussed briefly below. The first theory proposes that the persistence of a toxin depends on which target protein and where on that target protein that toxin attacks—Raciborska, et al. Can. J. Physiol. Pharmcol. 77:679-688 (1999). For example, SNAP-25 and VAMP are proteins required for vesicular docking, a necessary step for vesicular exocytosis. BoNT/A cleaves the target protein SNAP-25 and BoNT/B cleaves the target protein VAMP, respectively. The effect of each is similar in that cleavage of either protein compromises the ability of a neuron to release neurotransmitters via exocytosis. However, damaged VAMP may be more easily replaced with new ones than damaged SNAP-25, for example by replacement synthesis. Therefore, since it takes longer for cells to synthesize new SNAP-25 proteins to replace damaged ones, botulinum toxin type A has longer persistence.

Additionally, the site of cleavage by a toxin may dictate how quickly the damaged target proteins may be replaced. For example, botulinum toxin type A and E both cleave SNAP-25. However, they cleave at different sites and BoNT/E causes shorter-lasting paralysis in patients, compared with BoNT/A—id. at 685-6.

The second theory proposes that the particular persistence of a toxin depends on its particular intracellular half-life or stability, i.e. the longer the toxin is available in the cell, the longer the effect—Keller, et al. FEBS Letters 456:137-42 (1999). Many factors contribute to the intracellular stability of a toxin, but primarily, the better it is able to resist the metabolic actions of intracellular proteases to break it down, the more stable it is—Erdal, et al. Naunynschmiedeber's Arch. Pharmacol. 351:67-78 (1995).

In general, the ability of a molecule to resist metabolic actions of intracellular proteases may depend on its structures. For example, the primary structure of a molecule may include a unique primary sequence which may cause the molecule to be easily degraded by proteases or difficult to be degraded. For example, Varshavskv describes polypeptides terminating with certain amino acids as beinge more susceptible to degrading proteases—Proc. Natl. Acad. Sci. USA 93:12142-12149 (1996).

Furthermore, intracellular enzymes are known to modify molecules, for example polypeptides through, for example, N-glycosylation, phosphorylation etc.—this kind of modification will be referred to herein as “secondary modification”. “Secondary modification” often refers to the modification of endogenous molecules, for example, polypeptides after they are translated from RNAs.

However, as used herein, “secondary modification” may also refer to an enzyme's, for example an intracellular enzyme's, ability to modify exogenous molecules. For example, after a patient is administered with exogenous molecules, e.g. drugs, these molecules may undergo a secondary modification by the action of the patient's enzymes, for example intracellular enzymes.

Certain secondary modifications of molecules, for example polypeptides, may resist or facilitate the actions of degrading proteases. These secondary modifications may, among other things, (1) affect the ability of a degrading protease to act directly on the molecule and/or (2) affect the ability of the molecules to be sequestered into vesicles to be protected against these degrading proteases.

The Clostridial neurotoxin may be one of the botulinum toxin serotypes A, B, C₁, D, E, F and G, including a botulinum toxin which is free of the complexing proteins present in natural neurotoxin or a neurotoxin modified chemically or modified by genetic manipulation. The chemically or gentically modified neurotoxin is free of the complexing proteins which naturally form complexes with botulinum neurotoxin as well.

The modification of the neurotoxin derived from botulinum neurotoxin due to chemical modifying or genetic manipulation can occur on each part of the neurotoxin protein, for instance on the heavy chain part and/or on the light chain part of the neurotoxin molecule. There might be one modification or more modifications. In one embodiment, the heavy chain of the neurotoxin protein derived from botulinum neurotoxin comprises one or more modifications which may decrease or increase the affinity of the neurotoxin for binding to nerve cells when compared to the native neurotoxin. Such modified neurotoxin may comprise at least one substitution and/or deletion and/or insertion and/or addition and or posttranslational modification of amino acids of the neurotoxin and preferably of the heavy chain of the neurotoxin.

There is a need to have modified neurotoxins which have efficacies of the various botulinum toxin serotypes, but with altered (shorter) biological persistence and which exhibit reduced antibody formation.

### SUMMARY OF THE INVENTION

The present invention relates to enhancement of healing of injured surface or superficial tissue in a patient using naturally occurring and/or modified Clostridium neurotoxins, as well as those neurotoxins free of complexing proteins. Such use embraces applications in wound healing (which includes use in preventing scar formation) as well as use in ophthalmology (e.g. in treatment of injured corneal tissue, for example to close inflamed eyes). Their diagnostic usage is a further indication. Clostridium botulinum neurotoxins from serotypes A, B, C₁, D, E, F and G are contemplated for administration to facilitate wound healing and preventing scar formation according to the desired duration of effect. Moreover, Clostridial neurotoxins which have a short duration of action and which may be free of complexing proteins may be used where a relatively short duration of muscle paralysis is desired.

The invention is based on immobilizing the area around injured tissue such as a wound by paralysing the muscles acting thereon. This can be achieved by injecting a peripherally acting muscle relaxant directly into the appropriate muscles. The peripherally acting muscle relaxant is chosen from a natural or modified neurotoxin, such as a Clostridial neurotoxin, with a short duration of action and which may be free of complexing proteins. Botulinum toxins of type E and type F are embodiments of this invention.

The present invention also provides for improved healing in keratitis and certain operative interventions of the eye. Closure of the eyelids can be achieved by drug-induced ptosis which is achieved by administering a peripherally and locally acting muscle relaxant. This muscle relaxant is chosen from the natural or modified short-acting neurotoxins, such as a Clostridial neurotoxin, with a short duration of action and which may be free of complexing proteins. This measure serves to immobilize the eye and thus favors healing. Botulinum toxins of type E or type F are embodiments.

In another aspect of the invention, short-acting botulinum toxins which are free of complexing proteins are used as a diagnostic tool to localize the optimal area of injection for longer-acting botulinum toxins used to treat various conditions. Botulinum toxin type E is an embodiment.

What we therefore believe to be comprised by our invention may be summarized inter alia in the following words:

Use of a natural or modified Clostridium neurotoxin for the manufacture of a medicament for enhancing healing of injured surface or superficial tissue of a patient, wherein said medicament is manufactured for local administration into or in close proximity to said injured tissue, such a

use wherein the Clostridium neurotoxin is free of complexing proteins which naturally form complexes with Clostridial neurotoxins, such a

use wherein the natural or modified Clostridium neurotoxin is characterized by short-lasting efficacy of about 3 to 4 weeks, such a

use wherein the Clostridium neurotoxin is botulinum toxin type F, such a

use wherein the natural or modified Clostridium neurotoxin is characterized by short-lasting efficacy of about 3 to 10 days, such a

use wherein the Clostridium neurotoxin is botulinum toxin type E, such a

use wherein the Clostridium neurotoxin is a modified neurotoxin with an efficacy duration of about 1 to 4 weeks, such a

use wherein said injured tissue comprises a wound, such a

use wherein said injured tissue comprises corneal tissue and said medicament is manufactured for local administration into or in close proximity to the adjacent eyelid, and such a

use wherein a two component medicament is manufactured, the first component comprising a Clostridium neurotoxin having short-lasting efficacy of about 3 to 10 days for use in determining an optimal area for administration, and the second component comprising a Clostridium neurotoxin having long-lasting efficacy of about 12 weeks for subsequent therapeutic administration. Furthermore,

a method of treating a patient having a surface or superficial tissue injury, said method comprising locally administering a natural or modified Clostridium neurotoxin into or in close proximity to said injured tissue, such that healing of the injury is enhanced, such a

method wherein the Clostridium neurotoxin is free of complexing proteins which naturally form complexes with Clostridial neurotoxins, such a

method wherein the natural or modified Clostridium neurotoxin is characterized by short-lasting efficacy of about 3 to 4 weeks, such a

method wherein the Clostridium neurotoxin is botulinum toxin type F, such a

method wherein the natural or modified Clostridium neurotoxin is characterized by short-lasting efficacy of about 3 to 10 days, such a

method wherein the Clostridium neurotoxin is botulinum toxin type E, such a

method wherein the Clostridium neurotoxin is a modified neurotoxin with an efficacy duration of about 1 to 4 weeks, such a

method wherein said injured tissue comprises a wound, and such a

method wherein said injured tissue comprises corneal tissue and said Clostridium neurotoxin is administered into or in close proximity to the adjacent eyelid such that the eyelid remains closed and healing of the injured corneal tissue is enhanced. Moreover,

a method of treating a patient having an ophthalmic condition requiring closure of an eyelid for healing of the ophthalmic condition, comprising local administration of a natural or modified Clostridium neurotoxin in or in close proximity to the eyelid such that the eyelid remains closed and healing of the ophthalmic condition is enhanced. Additionally,

a method of determining an optimal area for injection of a Clostridium neurotoxin having long-lasting efficacy of about 12 weeks, comprising one or more initial local administrations of a natural or modified Clostridium neurotoxin having short-lasting efficacy of about 3 to 10 days in order to determine the effects of administration at a specific site or sites and thereby optimise the administration site to be used subsequently for said Clostridium neurotoxin having long-lasting efficacy, such a

method wherein the natural or modified Clostridium neurotoxin is characterised by short-lasting efficacy of about 3 to 10 days and is free of complexing proteins which naturally form complexes with Clostridial neurotoxins, and such a

method wherein the natural or modified Clostridium neurotoxin is botulinum toxin type E. Also,

a combined medicament comprising separately administrable first and second components wherein the first component comprises a Clostridium neurotoxin having short-lasting efficacy of about 3-10 days and the second component comprises a Clostridium neurotoxin having long-lasting efficacy of about 12 weeks, and such a

combined medicament further including instructions for use of said first component in determining the effects of administration to a patient at a specific site or sites so as to permit selection of an optimal site for subsequent administration of said second component.

### DETAILED DESCRIPTION OF THE INVENTION

As described herein, wound healing after injury or surgical intervention is adversely affected by tension on the wound margins. The present invention embraces enhancement of wound healing and prevention of scar formation using naturally occurring and/or modified neurotoxins, including Clostridial neurotoxins, as well as those neurotoxins which are free of complexing proteins. This aspect of the invention is based on a new method to immobilize the area around the wound by paralyzing the muscles acting on the wound. This can be achieved by injecting a peripherally acting muscle relaxant directly into the appropriate muscles. Conventional muscle relaxants are, however, unsuitable for this for two reasons. Firstly, due to their small molecular weights they rapidly diffuse outward from the site of injection, thus producing undesirable effects in other parts of the body. Secondly, they are metabolized locally very rapidly and thus lose their efficacy.

Botulinum toxin, a peripherally acting muscle relaxant, advantageously remains at the site of injection sufficiently long to be taken up by the nerves where it remains in its active form for a long period of time. Due to the toxin's high molecular weight, the amount not taken up diffuses only slowly out from the injection site. Because of its dilution in the circulating blood, more distant nerves are not affected. The toxin is quickly inactivated by proteases in the serum. The various serotypes of botulinum toxin have different durations of action. While serotypes A and B block nerves for many weeks, serotype F does so for 34 weeks and serotype E for only 3-10 days. To achieve a brief period of paralysis the toxin must be injected a few days before the operation at one or several sites around the operation field, depending on the size of the muscle to be paralyzed. Serotype E or F may be selected according to the desired period of paralysis. Since the musculature at the chosen operative site is already paralyzed by the toxin at the time of operation, the anaesthetist requires smaller amounts of postsynaptic acting muscle relaxants. The danger of postoperative respiratory impairment by paralysis of the respiratory muscles is thus reduced. As local paralysis at the site of operation is maintained for up to 4-5 days postoperatively, the wound sutures are subjected to no additional tension during this time. The period of local immobilization should be maintained maximal until the wound is completely healed, typically 1-2 weeks maximum. If wound healing is complicated, for example by secondary healing, paralysis lasting a longer period of time may be indicated. In another embodiment, where an extended duration of muscle paralysis in wound healing is desired, administration of botulinum toxin serotype A or B is warranted. Recovery of nerve function occurs slowly after breakdown of the toxins in the nerve cells and is complete approximately 2 days after full proteolytic degradation of the toxin. It is not expected that the brief immobilization leads to any significant atrophy of the muscle.

In another embodiment, Clostridium botulinum neurotoxins from serotypes A or B, C₁, D, E, F, G which are free of complexing proteins, hemagglutinins, and other exogenous proteins may be advantageously used to facilitate wound healing and prevent scar formation. As an alternative to the two commercial type A botulinum toxin complex products, BOTOX® and DYSPORT®, and also as alternative to the complexes described in the prior art of the other types (B, C₁, D, E, F, G), a novel pharmaceutical has been developed which comprises only neurotoxin (type A, B, C₁, D, E, F or G) free of complexing proteins, hemagglutinins and other exogenous proteins. Because of its lower molecular mass, it diffuses more quickly to the target cells in which it is taken up, before immune cells, attracted by hemagglutinins, are activated. Antigenicity studies demonstrate that neurotoxin of any type which is free of complexing proteins, induces no, or at the most very little, formation of antibodies, which is distinct from commercial products of type A and the complexes of types B to G. On therapeutic use of this newly developed pharmaceutical (neurotoxin of types A, B, C₁, D, E, F or G which is free of complexing proteins) there is no failure of therapy due to antibodies even after repeated administration. It has also been possible to show that such neurotoxins are, because of their immediate bioavailability, still suitable for the therapy of patients who have developed, after administration of a botulinum toxin complex, e.g. after treatment with BOTOX® or DYSPORT®, an antibody titer against the appropriate type (so-called secondary non-responders), that is to say are no longer amenable to further treatment with BOTOX® or DYSPORT®, because administration of the commercial toxins no longer provides therapeutic effect.

This newly developed pharmaceutical can be employed with particular advantage for patients who have never, or not for many years, been treated with botulinum neurotoxin, because their antibody titer is low or zero from the outset. The advantage of its use is then that the increase in the titer in these patients due to the treatment with pure toxin is zero, or at the most very insignificant. In other words, the newly developed therapeutic composition can be administered over long periods without losing its effect. It is also suitable for patients who exhibit an antibody titer against a botulinum toxin.

The induction of antibodies during therapy with a Clostridium botulinum neurotoxin is thus prevented by administering a neurotoxin free of complexing proteins in place of the high molecular weight toxin complexes. The neurotoxin which has been completely separated from the complex proteins is immediately bioavailable and can bind directly to the nerve endings of the motor endplates.

In keratitis and certain operative interventions on the eye, either a bandage is put over the eye or the upper and lower lids are sutured together to keep the eye closed. This measure serves to immobilize the eye and thus favors healing. Daily assessment of the healing process can be done at the time the bandage is changed. However, it is not possible to inspect the surface of the eye if the lids are sutured together. Closure of the eyelids can also be achieved by drug-induced ptosis, through the injection of a peripherally and locally acting muscle relaxant. Depending on the desired duration of closure, botulinum toxin E or F is injected into the levator palpebrae superioris muscle. The advantage of this procedure is obvious. The eye remains accessible for inspection so that the healing process can be monitored and any necessary further measures can be undertaken without stress to the patient. Transient ptosis is reversible, just like the paralyses described above.

As has been previously described in detail, botulinum toxin serotypes A and B are used in dystonia or spasticity of different origins. If the disorder is complex or if several muscle groups are involved in the symptoms, it is often not clear which muscle should be paralyzed by the toxin to provide maximal relief for the patient. Test injections of serotype A or B would cause additional stress to the patient if they were to be injected into the wrong muscle. To localize the optimal area of injection for toxin therapy with a long-lasting efficacy toxin, a test may be conducted using a toxin which exhibits a short duration of effect. Botulinum toxin E is suitable for such a diagnostic test. The patient can experience the expected changes before the actual treatment. As its action lasts for only a few days, the patient finds out what effects will be produced later by treatment with a long-lasting efficacy toxin. Should the injection not be optimal, the disturbing effect will last for only a short time and another injection can be performed to test the effect on another muscle.

### EXAMPLES

### Example 1

Enhanced Wound Healing by Botulinum Injection in Humans

A patient undergoes scar revision excision surgery. The scar is located on the abdomen. The scar was a result of a trauma, and was closed at a tertiary referral center at the time.

The patient is placed in a supine position, and 5 ml of 0.5% lidocaine with 1:200,000 epinephrine is locally injected. The scar is excised and bleeding is controlled with monopolar cautery. Botulinum toxin A, which is free of complexing proteins is injected (10 units) into the wound periphery and fanning out from the wound. The wound was closed using 6-0 Vicryl for deep and 6-0 Nylon for superficial sutures.

Approximately 24 hours after surgery, the patient develops marked paralysis of the injection muscles, and had lost the ability to move the skin in an area of about 4 cm in diameter around the excision. The wound heals well in the early postoperative period. It is apparent that there is decreased movement and tension on the wound edges. The wound of the patient heals without complications. Compared to the preoperative scar, the cosmetic appearance of the resulting scar 12 months postoperatively is excellent and superior to the initial scar.

### Example 2

Botulinum Induced Ptosis to Promote Corneal Healing

A patient suffers from keratitis or undergoes surgical intervention on the eye.

Botulinum toxin type E or F which is free of complexing proteins is injected into the levator palpebral superioris to produce a flaccid ptosis on the upper lid and provide safe and effective protection for the cornea. The eye is inspected to monitor the healing process. Injections are repeated until the underlying disease or condition heals.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

We claim:

1. A method of treating a patient having a surface or superficial tissue injury, said method comprising locally administering a natural or modified Clostridium neurotoxin into or in close proximity to said injured tissue, such that healing of the injury is enhanced.

2. The method of claim 1, wherein the Clostridium neurotoxin is free of complexing proteins which naturally form complexes with Clostridial neurotoxins.

3. The method of claim 1, wherein the natural or modified Clostridium neurotoxin is characterised by short-lasting efficacy of about 3 to 4 weeks.

4. The method of claim 3, wherein the Clostridium neurotoxin is botulinum toxin type F.

5. The method of claim 1, wherein the natural or modified Clostridium neurotoxin is characterised by short-lasting efficacy of about 3 to 10 days.

6. The method of claim 5, wherein the Clostridium neurotoxin is botulinum toxin type E.

7. The method of claim 1, wherein the Clostridium neurotoxin is a modified neurotoxin with an efficacy duration of about 1 to 4 weeks.

8. The method of claim 1, wherein said injured tissue comprises a wound.

9. The method of claim 1, wherein said injured tissue comprises corneal tissue and said Clostridium neurotoxin is administered into or in close proximity to the adjacent eyelid such that the eyelid remains closed and healing of the injured corneal tissue is enhanced.

10. A method of determining an optimal area for injection of a Clostridium neurotoxin having long-lasting efficacy of about 12 weeks, comprising one or more initial local administrations of a natural or modified Clostridium neurotoxin having short-lasting efficacy of about 3 to 10 days in order to determine the effects of administration at a specific site or sites and thereby optimise the administration site to be used subsequently for said Clostridium neurotoxin having long-lasting efficacy.

11. The method of claim 10, wherein the natural or modified Clostridium neurotoxin is characterised by short-lasting efficacy of about 3 to 10 days and is free of complexing proteins which naturally form complexes with Clostridial neurotoxins.

12. The method of claim 10, wherein the natural or modified Clostridium neurotoxin is botulinum toxin type E.

13. The method of claim 11, wherein the natural or modified Clostridium neurotoxin is botulinum toxin type E.

14. A kit comprising separately administrable first and second components wherein the first component comprises a Clostridium neurotoxin having short-lasting efficacy of about 3 to 10 days and the second component comprises a Clostridium neurotoxin having long-lasting efficacy of about 12 weeks.

15. The kit of claim 14 further comprising instructions for use of said first component in determining the effects of administration to a patient at a specific site or sites so as to permit selection of an optimal site for subsequent administration of said second component.
